(19) United States
(12) Patent Application Publication
(10) Pub. No.: US 2021/0012889 A1
(43) Pub. Date: Jan. 14, 2021

(54) SYSTEMS AND METHODS FOR MEDICAL RESOURCE MANAGEMENT

(71) Applicant: Avail Medsystems, Inc., Palo Alto, CA (US)

(72) Inventors: Daniel Hawkins, Palo Alto, CA (US); Ravi Kalluri, Palo Alto, CA (US); Christopher P. Gornick, San Francisco, CA (US)

(21) Appl. No.: 16/919,656

(22) Filed: Jul. 2, 2020

Related U.S. Application Data
(60) Provisional application No. 62/869,839, filed on Jul. 2, 2019.

(51) Int. Cl. G16H 40/20; G06Q 10/08; G06Q 10/10; G06K 7/10; G06K 9/00

(52) U.S. Cl. CPC G16H 40/20; G06Q 10/0875; G06K 2209/057; G06K 7/10366; G06K 9/00624; G06Q 10/10

(57) ABSTRACT

Described are platforms, systems, media, and methods for enterprise resource planning and tracking inventory. The systems and methods provided herein may be able to allow for items at a health care facility to be tracked in real-time. Item storage and item usage may be updated in real-time. In some instances, information associated with items may be accessed and presented to individuals who are managing or using the items.

### CROSS-REFERENCE

This application claims the benefit of U.S. Provisional Application No. 62/869,839, filed Jul. 2, 2019, which application is incorporated herein by reference in its entirety.

### BACKGROUND OF THE INVENTION

Inventories of many reusable and disposable medical equipment stored and used in hospitals are consigned to the medical facility by a third party vendor. Such vendors vie for shelf space within the hospital, and restock when necessary. Traditionally, vendor representatives physically visit the shelves at the hospitals to check inventory, which can be time consuming and costly.

Furthermore, hospitals often have contractual arrangements with vendors for the use of certain medical products for a given procedure or during a set time range. Information about usage of the medical products at hospitals is typically captured after a delay, and oftentimes medical personnel do not have visibility about the arrangements or product usage.

### SUMMARY OF THE INVENTION

A need exists for improved systems and methods of enterprise resource planning. A need exists for systems and methods that allow for equivalence and pricing notifications regarding reusable or disposable medical equipment. A further need exists for systems that monitor and communicate medical equipment usage and stock parameters to administrators and vendors.

One aspect, disclosed herein is a computer-implemented system comprising: a digital processing device comprising: at least one processor, an operating system configured to perform executable instructions, a memory, and a computer program including instructions executable by the digital processing device to create an enterprise resource planning application comprising: a communication module receiving a first item and a second item, wherein: the first item is associated with a first indicator, a first price structure or contractual term, a first item stock, a first item utilized quantity, and an equivalence association with the second item; and the second item is associated with a second price structure or contractual term, a second item stock, a second item utilized quantity, and an equivalence association with the first item; an inventory module receiving a practitioner item selection from a sensor, wherein the practitioner item selection is associated with the first indicator; an equivalence notification module displaying an equivalence notification to a practitioner, wherein the equivalence notification is associated with the second item based on the equivalence association of the first item with the second item and one or more of the first price structure or contractual term, the second price structure or contractual term, the first item stock, the second item stock, the first item utilized quantity, and the second item utilized quantity; and an equivalent item selection module receiving a practitioner response to the equivalence notification and updating the practitioner item selection data based on the practitioner response.

In some embodiments, the enterprise resource planning application further comprises a reporting module receiving the practitioner response and updating at least one of the first item stock, the second item stock, the first item utilized quantity, and the second item utilized quantity based on the practitioner response. In some embodiments, at least one of the first item and the second item comprise a tool, an instrument, an implant, a medicine, an ointment, or a practitioner device. In some embodiments, at least one of the first indicator and the second indicator comprise a barcode, a QR code, an image, a label, an icon, an alphanumeric identification, a weight, or any combination thereof. In some embodiments, the sensor comprises a camera, a video camera, an RFID reader, a radar, a sonar, a LIDAR, a thermal imaging camera, a scale, a button, or any combination thereof. In some embodiments, at least one of the first item and the second item comprise an equivalence association with 2 or more items. In some embodiments, the first item is further associated with the first item utilized quantity of prior period of time, wherein the second item is further associated with the second item utilized quantity of prior period of time, or both. In some embodiments, the inventory module further receives a practitioner item confirmation, a practitioner item note, or both, wherein the practitioner item confirmation and the practitioner item note are associated with the first indicator. In some embodiments, at least one of the first price structure or contractual term and the second price structure or contractual term comprise at least a primary cost per item for a primary quantity of items and a secondary cost per item for a secondary quantity of items. In some embodiments, at least one of the first price structure or contractual term and the second price structure or contractual term comprise a set price per part per quantity. In some embodiments, the equivalence notification comprises the first price structure or contractual term, the second price structure or contractual term, the first item stock, the second item stock, the first item utilized quantity, the second item utilized quantity, or any combination thereof. In some embodiments, the equivalence notification comprises a difference between the first price structure or contractual term and the second price structure or contractual term, the first item stock and the second item stock, the first item utilized quantity and the second item utilized quantity, or any combination thereof. In some embodiments, the equivalence notification comprises a quantity of the first item or the second item necessary to reach the secondary cost per item. In some embodiments, the equivalence notification comprises an interchangeability rating based on a historical usage data of the first item and the second item. In some embodiments, at least one of the first price structure or contractual term and the second price structure or contractual term comprise a lack of a price structure or contractual term. In some embodiments, the equivalence notification is further associated with contract met status based on the first price structure or contractual term and the first item utilized quantity, or the second price structure or contractual term and the second item utilized quantity.

Another aspect provided herein is a computer-implemented an enterprise resource planning method comprising: receiving, by a communication module, a first item and a second item, wherein: the first item is associated with a first indicator, a first price structure or contractual term, a first item stock, a first item utilized quantity, and an equivalence association with the second item; and the second item is associated with a second price structure or contractual term, a second item stock, a second item utilized quantity, and an equivalence association with the first item; receiving, by an inventory module, a practitioner item selection from a sensor, wherein the practitioner item selection is associated with the first indicator; displaying, by an equivalence notification module, an equivalence notification associated with the second item to a practitioner based on the equivalence association of the first item with the second item and one or more of the first price structure or contractual term, the second price structure or contractual term, the first item stock, the second item stock, the first item utilized quantity, and the second item utilized quantity; and receiving, by an equivalent item selection module, a practitioner response to the equivalence notification and updating the practitioner item selection data based on the practitioner response.

In some embodiments, the method further comprises receiving, by a reporting module, the practitioner response and updating at least one of the first item stock, the second item stock, the first item utilized quantity, and the second item utilized quantity based on the practitioner response. In some embodiments, at least one of the first item and the second item comprise a tool, an instrument, an implant, a medicine, an ointment, or a practitioner device. In some embodiments, at least one of the first indicator and the second indicator comprise a barcode, a QR code, an image, a label, an icon, an alphanumeric identification, a weight, or any combination thereof. In some embodiments, the sensor comprises a camera, a video camera, an RFID reader, a radar, a sonar, a LIDAR, a thermal imaging camera, a scale, a button, or any combination thereof. In some embodiments, at least one of the first item and the second item comprise an equivalence association with 2 or more items. In some embodiments, the first item is further associated with the first item utilized quantity of prior period of time, wherein the second item is further associated with the second item utilized quantity of prior period of time, or both. In some embodiments, the inventory module further receives a practitioner item confirmation, a practitioner item note, or both, wherein the practitioner item confirmation and the practitioner item note are associated with the first indicator. In some embodiments, at least one of the first price structure or contractual term and the second price structure or contractual term comprise at least a primary cost per item for a primary quantity of items and a secondary cost per item for a secondary quantity of items. In some embodiments, at least one of the first price structure or contractual term and the second price structure or contractual term comprise a set price per part per quantity. In some embodiments, the equivalence notification comprises the first price structure or contractual term, the second price structure or contractual term, the first item stock, the second item stock, the first item utilized quantity, the second item utilized quantity, or any combination thereof. In some embodiments, the equivalence notification comprises a difference between the first price structure or contractual term and the second price structure or contractual term, the first item stock and the second item stock, the first item utilized quantity and the second item utilized quantity, or any combination thereof. In some embodiments, the equivalence notification comprises a quantity of the first item or the second item necessary to reach the secondary cost per item. In some embodiments, the equivalence notification comprises an interchangeability rating based on a historical usage data of the first item and the second item. In some embodiments, at least one of the first price structure or contractual term and the second price structure or contractual term comprise a lack of a price structure or contractual term. In some embodiments, the equivalence notification is further associated with contract met status based on the first price structure or contractual term and the first item utilized quantity, or the second price structure or contractual term and the second item utilized quantity.

Another aspect provided herein is a non-transitory computer-readable storage media encoded with a computer program including instructions executable by a processor to create an application comprising: a communication module receiving a first item and a second item, wherein: the first item is associated with a first indicator, a first price structure or contractual term, a first item stock, a first item utilized quantity, and an equivalence association with the second item; and the second item is associated with a second price structure or contractual term, a second item stock, a second item utilized quantity, and an equivalence association with the first item; an inventory module receiving a practitioner item selection from a sensor, wherein the practitioner item selection is associated with the first indicator; an equivalence notification module displaying an equivalence notification to a practitioner, wherein the equivalence notification is associated with the second item based on the equivalence association of the first item with the second item and one or more of the first price structure or contractual term, the second price structure or contractual term, the first item stock, the second item stock, the first item utilized quantity, and the second item utilized quantity; and an equivalent item selection module receiving a practitioner response to the equivalence notification and updating the practitioner item selection data based on the practitioner response.

In some embodiments, the application further comprising a reporting module receiving the practitioner response and updating at least one of the first item stock, the second item stock, the first item utilized quantity, and the second item utilized quantity based on the practitioner response. In some embodiments, at least one of the first item and the second item comprise a tool, an instrument, an implant, a medicine, an ointment, or a practitioner device. In some embodiments, at least one of the first indicator and the second indicator comprise a barcode, a QR code, an image, a label, an icon, an alphanumeric identification, a weight, or any combination thereof. In some embodiments, the sensor comprises a camera, a video camera, an RFID reader, a radar, a sonar, a LIDAR, a thermal imaging camera, a scale, a button, or any combination thereof. In some embodiments, at least one of the first item and the second item comprise an equivalence association with 2 or more items. In some embodiments, the first item is further associated with the first item utilized quantity of prior period of time, wherein the second item is further associated with the second item utilized quantity of prior period of time, or both. In some embodiments, the inventory module further receives a practitioner item confirmation, a practitioner item note, or both, wherein the practitioner item confirmation and the practitioner item note are associated with the first indicator. In some embodiments, at least one of the first price structure or contractual term and the second price structure or contractual term comprise at least a primary cost per item for a primary quantity of items and a secondary cost per item for a secondary quantity of items. In some embodiments, at least one of the first price structure or contractual term and the second price structure or contractual term comprise a set price per part per quantity. In some embodiments, the equivalence notification comprises the first price structure or contractual term, the second price structure or contractual term, the first item stock, the second item stock, the first item utilized quantity, the second item utilized quantity, or any combination thereof. In some embodiments, the equivalence notification comprises a difference between the first price structure or contractual term and the second price structure or contractual term, the first item stock and the second item stock, the first item utilized quantity and the second item utilized quantity, or any combination thereof. In some embodiments, the equivalence notification comprises a quantity of the first item or the second item necessary to reach the secondary cost per item. In some embodiments, the equivalence notification comprises an interchangeability rating based on a historical usage data of the first item and the second item. In some embodiments, at least one of the first price structure or contractual term and the second price structure or contractual term comprise a lack of a price structure or contractual term. In some embodiments, the equivalence notification is further associated with contract met status based on the first price structure or contractual term and the first item utilized quantity, or the second price structure or contractual term and the second item utilized quantity.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a non-limiting schematic diagram of an enterprise resource planning application, according to some embodiments herein;

FIG. 1B shows an exemplary system where an enterprise resource planning application, according to some embodiments herein;

FIG. 2 shows an image of an exemplary first enterprise resource planning system, according to some embodiments herein;

FIG. 3 shows an image of an exemplary second enterprise resource planning system, according to some embodiments herein;

FIG. 4 shows a non-limiting schematic diagram of a real-time inventory capture system, according to some embodiments herein;

FIG. 5 shows a non-limiting schematic diagram of a digital processing device; in this case, a device with one or more CPUs, a memory, a communication interface, and a display, according to some embodiments herein;

FIG. 6 shows a non-limiting schematic diagram of a web/mobile application provision system; in this case, a system providing browser-based and/or native mobile user interfaces, according to some embodiments herein; and

FIG. 7 shows a non-limiting schematic diagram of a cloud-based web/mobile application provision system; in this case, a system comprising an elastically load balanced, auto-scaling web server and application server resources as well synchronously replicated databases, according to some embodiments herein.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention provides systems and methods for communication facilitation. Various aspects of the invention described herein may be applied to any of the particular applications set forth below. The invention may be applied as a part of a health care scheduling system or communication system. It shall be understood that different aspects of the invention can be appreciated individually, collectively or in combination with each other.

Tracking of reusable and disposable equipment is key towards surgery planning, inventory assessment, billing, and sales monitoring. Capturing of real-time utilization data enables the selection of the functionally equivalent but more economic tools and disposables. Further, such real-time monitoring enables automatic live inventory tracking for hospital stocking and billing, and live current usage statistics for device sales organizations.

### Enterprise Resource Planning Application

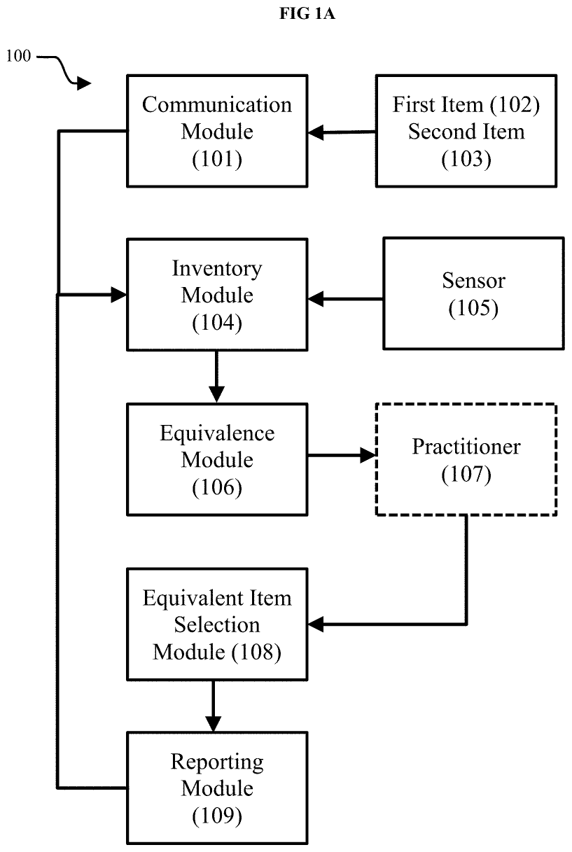

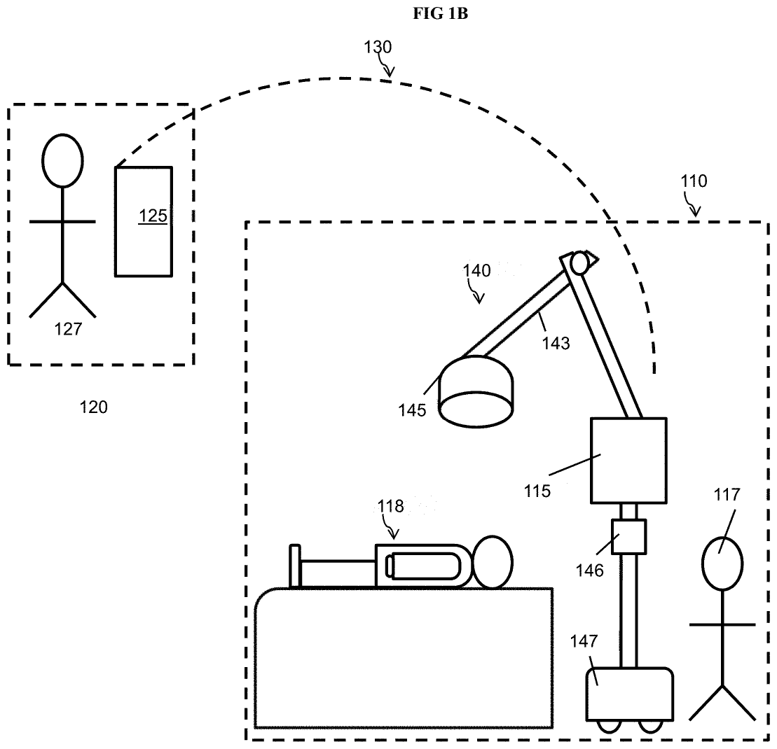

FIG. 1A shows a non-limiting schematic diagram of an enterprise resource planning application comprising a communication module 101 receiving data relating to one or more items, such as a first item 102 and a second item 103, an inventory module 104 receiving a practitioner item selection from a practitioner 107, an equivalence notification module 106 displaying an equivalence notification, an equivalent item selection module 108 receiving a practitioner response, and a reporting module 109 receiving the practitioner response. Such modules may be provided by way of example. The modules may include one or more software or hardware modules that perform the functionality. One or more steps or functionality provided herein may be performed by any number or combinations of modules.

The communication module 101 may comprise a digital processing device, a mobile device, a mobile application, a standalone application, or a web browser plug-in as described below. The communication module may provide an interface or portal for a user to interact with an enterprise resource planning system. The communication module may allow a user interface to be presented to a user that may allow a user to interact with the system. The communication module may optionally receive data from a user. The communication module may receive data with aid of a touchscreen, keys, mouse, stylus, touchpad, microphone, camera, scanner, infrared sensor, radiofrequency sensor, or any other type of user interactive device. The communication module may access data stored in one or more memory storage units.

The communication module 101 may obtain information about one or more items. Items may include one or more medical products. For example, at least one of the first item 102 and the second item 103 may comprise a tool, an instrument, an implant, a medicine, an ointment, or a practitioner device. At least one of the first item 102 and the second item 103 may include tools, instruments, implants, prostheses, disposables, or any other apparatus, appliance, software, or materials that may be intended by the manufacturer to be used for human beings. At least one of the first item 102 and the second item 103 may be used for diagnosis, monitoring, treatment, alleviation, or compensation for an injury or handicap. At least one of the first item 102 and the second item 103 may be used for diagnosis, prevention, monitoring, treatment, or alleviation of disease. In some instances, At least one of the first item 102 and the second item 103 may be used for investigation, replacement, or modification of anatomy or of a physiological process. Some examples of the first item 102 and the second item 103 may range from surgical instruments (e.g., handheld or robotic), catheters, endoscopes, stents, pacemakers, artificial joints, spine stabilizers, disposable gloves, gauze, IV fluids, drugs, and so forth. The enterprise resource planning application may receive and/or monitor 2, 3, 4, 5, 6, 7, 8, 9, 10, 20, 30, 40, 50, 100, 200, 300, 400, 500 or more items, including increments therein. Any description of an item may apply to any type of medical device, product, instrument, disposable, implant, medicine, and so forth. Any description of an item may be any type of medical resource, such as an object that may be used during a medical procedure.

An item, such as a first item or second item, may have an indicator. A single item may have a single indicator or multiple indicators. In some instances, each item may have one or more corresponding indicators. An indicator may or may not be unique to an item. The indicator may be unique to an item type or batch. The indicator may be unique to items provided by a particular vendor. In some embodiments, at least one of the first indicator and the second indicator comprise a barcode, a QR code, an RFID code, an image, a label, an icon, an alphanumeric identification, a weight, or any combination thereof. An indicator may be visually detectable or discernible. The indicator may be viewable with the naked eye. The indicator may be detectable under visible light, ultraviolet light, infrared light, or any other portion of an electromagnetic spectrum. The indicator may be provided on an outer surface of an item. The indicator may be provided on an outer surface of packaging for an item. At least one item, such as at least one of the first item 102 and the second item 103 may be further associated with corresponding item information. Examples of item information may include a purchase date, a use-by date, an expiration date, or any combination thereof. Further examples of item information may include corresponding vendor information, item name or type, item batch number, unique item identifier, conditions under which an item should be stored (e.g., temperature conditions, lighting conditions), or other information. The indicator itself may encode such information directly or indirectly. For instance, such information may be visually encoded in the indicator so that scanning or reading the indicator automatically provides access to the information. In some instances, the indicators may allow the system to access a memory, which stores the related information. For example, the indicators may be a unique identifier for the medical product, and the unique identifier may be used to access memory that stores information associated with the unique identifier.

In some instances, the communication module may obtain information about an item with aid of one or more user interactive devices. For example, a camera, scanner, microphone, radiofrequency detector, or any other type of sensor or user interactive device may collect information about the item. Any type of sensor may be utilized to collect data about an item. The sensor may be any type of imaging device, such as a visual camera, video camera, or IR camera. The sensor may include ultrasonic sensors, lidar, audio sensors (e.g., microphone), or any other type of sensor. A user may input the information directly by entering information about the item. A user may actively cause the communication module to obtain the information (e.g., the user enters information, the user holds the item to a scanner, the user takes a picture of the item with a camera, the user brings the item into a proximity of a sensor). The system may passively collect information about the item. For example, cameras may be positioned within the environment that automatically collect images and keep track of the items.

Data about an item may be collected with aid of a sensor. The data about the item may be received and the item may be recognized by the system. In some instances, the sensor may collect information about an item indicator. In some instances, the sensor may collect information about the item itself without requiring an indicator. For instance, one or more images of the item may be captured. The item may be recognized based on image recognition techniques. In some instances, machine learning techniques may be used to aid in identification of the item. In some embodiments, the machine learning may utilize deep convolution neural networks/Faster R-CNN Nast NasNet (COCO). The machine learning may utilize any type of convolutional neural network (CNN) and/or recurrent neural network (RNN). Shift invariant or space invariant neural networks (SIANN) may also be utilized. Image classification, object detection and object localization may be utilized. Any machine learning technique known or later developed in the art may be used. For instance, different types of neural networks may be used, such as Artificial Neural Net(ANN), Convolution Neural Net (CNN), Recurrent Neural Net (RNN), and/or their variants.

In some instances, the item may be recognized based on the indicator, such as a visual graphical code.

The first item 102 may be associated with a first indicator, a first price structure or contractual term, a first item stock, a first item utilized quantity, and/or an equivalence association with the second item 103. The second item 103 may be associated with a second price structure or contractual term, a second item stock, a second item utilized quantity, and/or an equivalence association with the first item 102. At least one of the first item 102 and the second item 103 may comprise an equivalence association with 2 or more items. Optionally, equivalence associations may be provided between items that have clinical equivalence. Optionally, equivalence associations may be provided by a clinician, a doctor, a hospital administrator, a supplier or any combination thereof. The equivalence may be determined by the Food and Drug Administration (FDA) or other agency. In some instances, a group of practitioners may determine clinical equivalence. Optionally, a group of practitioners or administration for a health care facility may make the determination of clinical equivalency for that particular health care facility. In some instances, a recognized standard of clinical equivalency may be determined by practitioners for particular items. Determinations of clinical equivalency may inform a database that may include relevant pricing and other contractual information for notifications.

Such items that have been determined to have clinical equivalency may be interchangeably used to yield the same or similar clinical outcomes. In some instances, multiple vendors may have products of the same type. These products may work equally well under certain circumstances. For example, a pacemaker A supplied by Vendor X may have clinical equivalency with a pacemaker B supplied by Vendor Y for a patient with certain physical conditions. In some instances, items may be clinically equivalent for all applications or conditions. In some instances, items may only be clinically equivalent under certain limited applications or conditions and may not be clinically equivalent under other conditions. The systems and methods provided herein may automatically track when items are clinically equivalent. The systems and methods provided herein may be able to determine whether items are clinically equivalent for a particular patient, based on the patient's condition. The patient's medical records may or may not be accessed by the system when determining clinical equivalency.

In some embodiments, the first item 102 is further associated with a first item utilized quantity, and wherein the second item 103 is further associated with a second item utilized quantity. The utilized quantity may refer to a utilized quantity over a set period of time. The prior period of time may be about 1 day, 2, days, 3 days, 5 days, 10 days, 15 days, 20 days, 30 days, 50 days, 100 days, 200 days, 300 days, or more.

Contractual arrangements may be made between health care facilities and/or a medical personnel or administrator and one or more vendors for medical products and/or support. The health care facility may comprise a hospital, a clinic, a shelter, a fire department, a police department, a military organization, or a disaster relief organization. The personnel or administrator may comprise a manager, coordinator, practitioner or any combination thereof at the above mentioned facilities. Contracts between health care facilities and vendors for medical products may include cost tiers and/or structures. In one example, a vendor may charge a health care facility a certain price per unit of a medical product. In turn, the health care facility may guarantee at least a certain percentage or usage level of the medical product. For instance, the health care facility may guarantee to use at least X number of units of the medical product per month, or other period of time. In another example, the health care facility may guarantee that at least Y % of a particular type of product used will include a vendor product per month, or other period of time. In some embodiments, if the health care facility doesn't meet a threshold (e.g., percentage or quantity), a different price per unit may apply. Any number of thresholds and corresponding pricing structures may be provided. As such, at least one of the first price structure or contractual term and the second price structure or contractual term may comprise at least a primary cost per item for a primary quantity of items and a secondary cost per item for a secondary quantity of items. In some embodiments, at least one of the first price structure or contractual term and the second price structure or contractual term comprise a set price per part per quantity. At least one of the first price structure or contractual term and the second price structure or contractual term may comprise a lack of a price structure or contractual term. In some embodiments, at least one of the primary quantity of items and the secondary quantity of items comprises a first item utilized quantity or a second item utilized quantity. In some embodiments, at least one of the first item utilized quantity or the second item utilized quantity is dynamic. In some embodiments, at least one of the first item utilized quantity or the second item utilized quantity is updated in real-time. In some embodiments, at least one of the first item utilized quantity or the second item utilized quantity used to test against the contractual terms for that item. In some embodiments, at least one of the first item utilized quantity or the second item utilized quantity represents a tally of products used over a certain period. In some embodiments, at least one of the first item utilized quantity or the second item utilized quantity is cleared after the certain period of time.

The systems and methods provided herein may advantageously track usage of medical products in real-time or substantially in real-time. For instance, the systems and methods provided herein may track that a product has been used within 4 hours, 3 hours, 2 hours, 1 hour, 30 minutes, 20 minutes, 15 minutes, 10 minutes, 5 minutes, 3 minutes, 2 minutes, 1 minute, 30 seconds, 20 seconds, 10 seconds, 5 seconds, 3 seconds, or 1 second of the product being used, the product being scanned, or being selected by a medical personnel to be used. The systems and methods may also store information about the contractual arrangements and keep in track in real-time or substantially real-time how product usage relates to the contractual arrangements. For example, the systems and methods may be able to determine in real-time when one or more thresholds for a particular medical product has been met. The system and method may be able to also keep track of corresponding pricing structures for a healthcare facility and one or more medical products provided by one or more vendors. In some instances, different medical products provided by different vendors to a particular healthcare facility may have clinical equivalency. The systems and methods may track multiple products having clinical equivalency. The systems and methods may be able to provide suggestions for product usage to provide an optimized or reduced price for a particular healthcare facility. The systems and methods may take multiple contractual arrangements with multiple vendors into account when determining a product suggestion or information on how to reduce or optimize pricing for a particular healthcare facility. Such considerations may take into account whether products have met the threshold for contractual obligations or not, and the thresholds for multiple contracts may be taken into account when determining pricing ramifications for a particular health care facility. The considerations and factors may be predetermined or may be adjustable. In some instances, a practitioner or administrator at a health care facility may adjust one or more factors that may affect product recommendations and/or equivalency notifications that are provided.

The equivalence notification module 106 may display an equivalence notification to a practitioner. The equivalence notification may be associated with the second item 103 based on the equivalence association of the first item 102 with the second item 103 and one or more of the first price structure or contractual term, the second price structure or contractual term, the first item stock, the second item stock, the first item utilized quantity, and the second item utilized quantity. An exemplary first price structure or contractual term comprises a formula of cost is equal to a base constant minus a quantity multiplied by an upscale constant. At least one of the base constant, the quantity and the upscale constant may be updated in real-time. The equivalence notification may be associated with two or more items. The equivalence notification may comprise a rank for each of the two or more items. The rank may be based on a price, a price difference, an efficacy, a stock, or any combination thereof. The equivalence notification may comprise a price, a price difference, an efficacy, a stock, or any combination thereof of the two or more items. The notification may or may not be based on a manufacturer price. The notification may or may not be based on a price for the particular healthcare facility based on contractual agreements between the healthcare facility and a vendor. The notification may recommend a second product that is clinically equivalent to a first product and for which the pricing to the healthcare facility is lower than for the first product when the first product is ‘not on contract’ with the vendor, or when a first product ‘has already met contractual obligations’, or the second product ‘has not yet met facility-vendor contract requirements’. Different prioritizations may be specified. In some instances, system-wide prioritizations for the rankings may be implemented. Alternatively, each health care facility may specify how recommendations/notifications are provided in ranking the products. As described elsewhere herein, rankings may be based on any factor or set of factors (e.g., efficacy, availability, absolute price, contractual price, overall cost to health care facility, existence or non-existence of contract, whether contractual obligations have been met, whether contractual obligations haven't been met, physician preference, group preference), and such factors may be weighted or prioritized (e.g., partially prioritized, absolutely prioritized). For instance, one, two, three, four, five or more factors may be considered and/or weighted when ranking products and/or making recommendations in a notification.

For example, when a medical practitioner is performing a medical procedure and selects an item, a notification may be displayed that other clinical equivalent products are available. In some instances, the notification is displayed only when most cost effective clinically equivalent items are available. If the item selected by the medical practitioner is the most cost effective, then the notification need not be displayed. Alternatively, clinically equivalent items may be displayed regardless. In some instances, multiple clinical equivalent items may be displayed. In some instances, they may be displayed in order of cost effectiveness (where the most cost effective option is displayed first or have the higher ranking) or any other factor. In some instances, various equivalent items may be displayed without the medical practitioner having made a selection. In some instances, a medical practitioner may request options for a particular type of item. The clinically equivalent items may be displayed in response to the request. The clinically equivalent items may be displayed in accordance with a ranking according to one or more factors, such as price or minimum thresholds.

In some embodiments, the equivalence notification comprises the first price structure or contractual term, the second price structure or contractual term, the first item stock, the second item stock, the first item utilized quantity, the second item utilized quantity, or any combination thereof. The practitioner item selection may be associated with the first indicator. In some embodiments, the inventory module 104 further receives a practitioner item confirmation, a practitioner item note, or both, wherein the practitioner item confirmation and the practitioner item note are associated with the first indicator. In some embodiments, the equivalence notification comprises a difference between the first price structure or contractual term and the second price structure or contractual term, the first item stock and the second item stock, the first item utilized quantity and the second item utilized quantity, or any combination thereof. In some embodiments, the equivalence notification comprises a quantity of the first item 102 or the second item 103 necessary to reach the secondary cost per item. In some embodiments, the equivalence notification comprises an interchangeability rating based on a historical usage data of the first item 102 and the second item 103. The equivalence notification may further comprise an expiration of the first item 103, the second item 103, or both. The equivalence notification may further comprise a statistic associated with a percentage and/or a quantity of positive patient outcomes associated with the first item, the second item, or both. The equivalence notification may further comprise a part quantity required to reach an economic incentive milestone, a minimum use threshold, or both. The equivalence notification may further prompt the practitioner to provide a specific non-interchangeability justification for using the first item 102 when the notification recommends the second item 103. This non-interchangeability justification may be forwarded to an administrator, a buyer, a seller, a vendor, or any combination thereof. In some embodiments the interchangeability rating is based on the non-interchangeability justification. The equivalence notification may be based on a saved preference for the first item 102 or the second item 103 associated with practitioner profile. The equivalence notification may be based on an ICD-9 code of the International Statistical Classification of Diseases and Related Health Problems.

For instance, particular practitioners may have preferred items for particular applications. The system may be aware of practitioner preferences. For instance, a practitioner may enter the practitioner's preference for particular types of items. For example, a practitioner may prefer a catheter from Vendor X. In other instances, the system may determine a practitioner's preferences based on historical usage. For example, if the practitioner consistently selects an item from Vendor X over an item from Vendor Y for a particular product type, the system may store information that the Vendor X item is preferred for that product type. When a particular practitioner is utilizing the system, the system may automatically inform the practitioner whether a practitioner's preferred item is available and/or make suggestions about clinically equivalent items.

Practitioners may or may not be provided with personal incentives to select particular items. For instance, if a particular product is more cost effective, this may translate to personal financial benefit for the practitioner when the practitioner selects the most cost effective option. Other types of incentives, such as improvements in ratings, benefits, prizes, may be provided to a practitioner to select a more cost effective option, or other item that meets a particular factor. The practitioner may or may not be made aware of incentives when selecting particular items. Practitioners may or may not be aware of benefits or incentives to the health care facility or group when selecting particular items. Practitioners may be made aware of directives by administrators. In some instances, directives by administrators may be applied to the system, and only items that meet the parameters set up by the administrators may be made available to practitioners. Such item availability may be updated in real-time as product usage relative to various parameters may be changed in real-time.

The inventory module 104 may receive data pertaining to a practitioner item selection from a sensor. The inventory module 104 may receive a sensor data from the sensor and determine the practitioner item selection based on the sensor data. In some embodiments, the sensor comprises a camera, a video camera, an RFID reader, a radar, a sonar, a LIDAR, a thermal imaging camera, a scale, a button, or any combination thereof. A sensor comprising a camera or a video camera may be configured to zoom in and out, pan, tilt, translate, or any combination thereof. Such sensors may be controlled directly by a human or through an autonomous algorithm. The data feed from the sensor may be viewable by an administrator, a vendor, or both. The video or image captured by the sensor may be modifiable to allow a user to zoom, fast forward, reverse, etc. The inventory module 104 may receive data pertaining to the practitioner item selection before, during, or after a medical procedure which employs the first item or the second item. The inventory module 104 may receive data pertaining to the practitioner item selection during the planning of the medical procedure which employs the first item or the second item. The equivalence notification module may display the equivalence notification during, or after a medical procedure which employs the first item or the second item. The equivalence notification module may display the equivalence notification the planning of the medical procedure which employs the first item or the second item.

The systems, methods, and applications herein may be able to simultaneously employ 2, 3, 4, 5, 6, 7, 8, 9, 10, 20, 50, or more sensors. The systems, methods, and applications herein may be able to simultaneously employ 2, 3, 4, 5, 6, 7, 8, 9, 10, 20, 50, or more displays. The systems, methods, and applications herein may be able to simultaneously monitor items in 2, 3, 4, 5, 6, 7, 8, 9, 10, 20, 50, or more storage units. The systems, methods, and applications herein may be configured for 24-hour monitoring over a period of time of 2, 3, 4, 5, 6, 7, 8, 9, 10, 20, 50, or more days.

The equivalent item selection module 108 may receive a practitioner response to the equivalence notification and update the practitioner item selection data based on the practitioner response. The practitioner response may comprise the practitioner removing the first item, the second item or both, expressing a voice command, providing a physical input, scanning the first item, scanning the second item, or any combination thereof. The physical input may comprise a key input, a touchscreen input, a swipe, a thumb print, a fingerprint, a gesture, or any combination thereof. In some instances, a practitioner may interact with a system without having to physically touch the system. This may advantageously allow the interactions to maintain sterility. For instance, a practitioner may use a voice command to automatically select a second item, which may automatically cause the system to update the records to include the second item and remove the first item. In another example, a practitioner may open a second item, or may scan a second item using a scanner or camera, which may cause the system to update the records to include the second item and remove the first item.

In some embodiments, a practitioner response may include the practitioner item selection. For instance, if a first product was initially planned for the procedure, but then a practitioner selects a second product instead, a record may be updated to indicate that the second product was used for the procedure. Similarly, the first product may be removed from a list of products used for the procedure. In some instances, the practitioner may select the second product based on a recommendation from a system.

The reporting module 109 may receive the practitioner response and update at least one of the first item stock, the second item stock, the first item utilized quantity, and the second item utilized quantity based on the practitioner response. The reporting module 109 may further transmit at least one of the first item stock, the second item stock, the first item utilized quantity, and the second item utilized quantity to a manufacturing representative, a marketing representative, a billing representative, a service representative, a supplier representative, a clinic/hospital department head/coordinator, or any combination thereof.

In some instances, the system may utilize information about the procedure to prospectively make one or more item recommendations. For instance, the system may recognize an ICD-9 code. The system may, based on the ICD-9 code, make a recommendation to a practitioner to consider using a first item from a first vendor. The recommendation may be based on any of the factors or combination of factors described elsewhere herein. The recommendation may be made to the practitioner prior to the practitioner making a product choice. For instance, a final stent choice may not happen during a portion of a procedure where the practitioner is assessing the arterial blockage using angiography. At that point, the system may suggest a first stent from a first vendor. The practitioner may make a choice to follow the suggestion or to select a different item (e.g., second stent). The system would recognize based on the item that is subsequently scanned, whether the recommended item was selected or not. For example, if the practitioner selects the first stent, the system would recognize that the product being scanned matches the recommendation. A notification may optionally be provided in the form of confirmation of the product being scanned. In another example, a practitioner may select a second stent, and the system would recognize that the product being scanned differs from the recommendation. A notification may optionally be provided that the product being scanned is different from the recommended product, or may provide an indication of the product scanned. The system may track which item is selected and used, and may optionally remove any previous items that are not used.

FIG. 1B shows an exemplary system where an enterprise resource planning application. In some instances, a practitioner 117 or other individual may be provided at a health care facility. The practitioner may perform a medical procedure on a patient 118. The practitioner and patient may be within a first location 110, such as an operating room, surgical suite, or other location or region at a health care facility. One or more cameras or other devices or sensors, such as those described elsewhere herein, may be provided at the first location. In some instances, one or more cameras may be supported by a medical console 140. The medical console may have a fixed location or may be able to move from location to location. For instance, the medical console may comprise a wheeled base 147 that may allow the medical console to be wheeled from location to location. The wheels may be locked into place at desired locations.

The medical console may comprise one or more arms 143. An arm may support one or more cameras 145 or other devices or sensors. An arm may be movable. For instance, an arm may comprise one, two, three, four, or more sections that may be movable relative to one another. A practitioner or other individual may be able to position the arm as desired. When positioned, the arm may remain stable and steady. A camera or other sensor supported by the arm may capture information, such as images, about a patient, such as a surgical site. Optionally, one or more other cameras 146 may be provided on a medical console. A camera, or other sensor, of a medical console may be used to capture images of medical products used. For instance, a camera may capture an image of a medical product that is used for a medical procedure. A practitioner may hold a medical product to a camera or other sensor to be scanned and identified. A camera or other sensor may capture data about an indicator of an item. For example, a camera may capture an image of a visual indicator, such as a barcode or QR code. The practitioner may or may not provide any other indication when the item is used. In some instances, capturing an image of an item may automatically indicate that an item is used for the procedure. Alternatively, the practitioner may provide a separate input when an item is actually used for the procedure. For instance, the practitioner may provide an input to a user device 115 when the item is used.

A user device 115 may optionally be provided on a medical console, or may be provided separately. The user device may display notifications to a practitioner. For example, the user device may provide information about items selected or used, or about available clinically equivalent items. The user device may optionally display information relating to contractual arrangements for particular items. The user device may display recommended items to use for a particular procedure or portion of the procedure.

The user device may be a tablet, computer (e.g., laptop, desktop), smartphone, personal digital assistant, or any other device that may be capable of network communications. The user device may communicate with one or more other remote devices 125. The communications 130 may occur directly or over a network. A remote user 127 may interact with the remote device. In some instances, data from the user device may be used to update information stored in a remote device (e.g., database) and/or cloud computing infrastructure. The remote user may be able to access the information.

For example, product usage information may be updated in substantially real-time. Such information may be made accessible to vendors and/or vendor representatives, hospital administrators, group members, other practitioners, insurance companies, electronic resource planning systems, manufacturers, marketing groups, and/or customer service/billing departments. Individuals associated with such entities may be able to access the information in substantially real-time. Providing the real-time capture of product usage may advantageously allow inventory to be tracked with a high degree of precision. Such information may also be useful for business systems, allowing for real-time utilization data from the operating room to affect upstream and/or downstream processes. For instance, real-time usage information may be advantageous to make adjustments to supply-chains as needed. For instance, if a manufacturer sees a trend for particular items, the manufacturer can make adjustments more rapidly. In another example, if particular products are used, vendor representatives for those particular products may be informed in real-time that they may need to provide present or future support for the product, which assists in allocation of workforce. The real-time capture of product usage may also have more instantaneous effects, such as noticing that a particular supply needs to be restocked more quickly. Such information may be presented to the relevant associated parties.

Items that are used at health care facilities are often stored ahead of time. For instance, one or more health care facilities may have a supply of particular products. At hospitals, there may be supply rooms or closets. These supply rooms may be separate from a location where the products will be used (e.g., operating room). In some instances, the products may be stored close to a location where the products will be used (e.g., within an operating room, but at a particular cabinet or location within the room). The supply locations may include one or more shelves for storing products. Vendors may reserve a particular amount of shelf space. In some instances, vendors may bid for quantities of shelf space. Vendor representatives may aid in making sure the shelves are stocked. Enterprise resource planning systems may aid in managing product inventory at health care facilities.

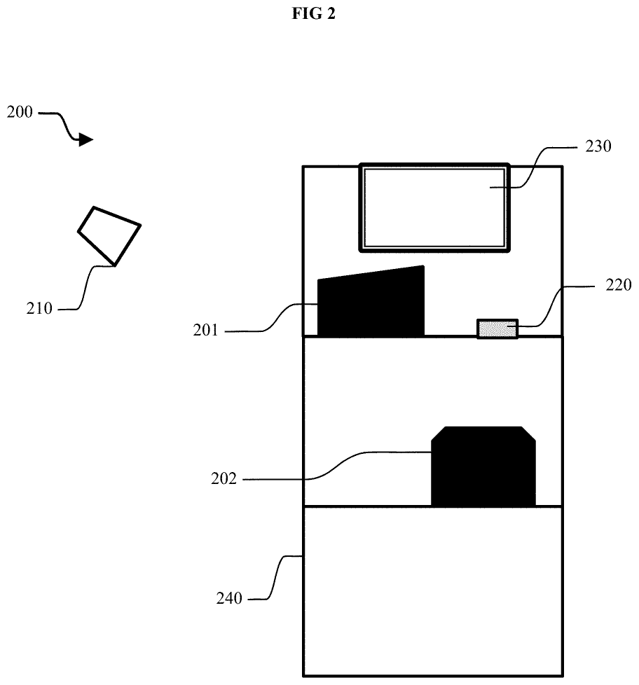

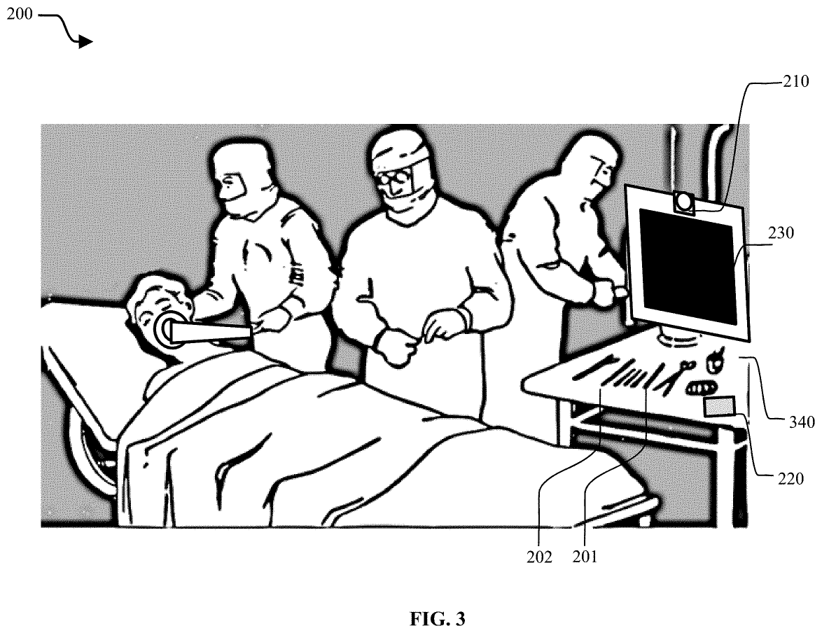

FIGS. 2-3 show images of an exemplary first and second enterprise resource planning system 200, respectively. As seen in FIGS. 2-3, the first enterprise resource planning system 200 may comprise one or more sensors, such as a first sensor 210 and/or a second sensor 220, a display 230, and/or a storage unit 240. At least one sensor, such as at least one of the first sensor 210 and the second sensor 220, may comprise a camera, a video camera, an RFID reader, a radar, a sonar, a LIDAR, a thermal imaging camera, a scale, a button, or any combination thereof. At least one of the first sensor 210 and the second sensor 220 may be configured to measure a presence or an absence of an item, such as the first item 201, the second item 202, or both. One or more sensors may be able to detect an indicator of an item. For instance, at least one of the first sensor 210 and the second sensor 220 may be configured to determine the first indicator of the first item 201, the second indicator of the second item 202, or both.

Optionally, a display may be provided. The display 230 may be configured to display information relating to items. For example, the display may display an equivalence notification to the practitioner. The display 230 may be further configured to display 230 one or more of the first indicator, the first price structure or contractual term, the second indicator, the second price structure or contractual term, the first item 201 stock, the second item 202 stock, the first item 201 utilized quantity, the second item 202 utilized quantity, and the practitioner response. The display may include information, such as alerts when supply of particular items is running low or about to expire.

This display may be shown on a screen. A graphical user interface (GUI) may be provided that may show the information, such as equivalence notification. The equivalence information may include any of the factors pertaining to the first or second items as described herein. The equivalence information may include information about why certain recommendations may be made. The thresholds for determining the recommendations may be displayed and/or adjustable by a practitioner or an administrator at a health care facility.

The storage unit 240 may comprise a bench, a cabinet, a shelf, a drawer, or a closet. One or more items may be stored in the storage unit.

FIG. 2 shows an exemplary embodiment, when the storage unit 240 comprises one or more shelves. The storage unit may be provided in a storage room or other location, as described. The one or more sensors may be integrated into the storage unit, supported by the storage unit, or separate from the storage unit. In one example, a sensor may comprise a camera that is capturing an image of at least a portion of the storage unit. The camera may be able to capture an image of the storage unit in its entirety, a shelf of the storage unit, or a portion of the storage unit or shelf. In some embodiments, the camera may be mounted on a wall, ceiling, furniture or location of a room where a storage unit is provided. The camera may be able to capture images of items and/or indicators of items. A sensor may capture information about all of the items in a storage unit or on a shelf at a time. For instance, a camera may capture an image of the entirety of the storage unit or shelf and be able to process information about the indicators from the items within the image. In some instances, a sensor may scan a portion of the storage unit or shelf at a time. For example, a camera may be able to scan along a shelf and then move on to another shelf to capture information about a subset of the items at a time.

The sensor may automatically operate to collect information about the items in the storage unit. For example, the sensor may automatically scan in accordance with a schedule or predetermined path to sense indicators of the various items. In one example, a camera may have a schedule where the camera scans a particular shelf from end to end, and then moves to another shelf from end to end, and so forth. The cameras may be constantly scanning, or may scan at selective periods according to a schedule. Computer control of the cameras may take place to automatically scan shelves on a periodic basis to provide update on items, or in response to particular procedures or protocols.

A sensor may be remotely controlled to collect information about the items in the storage unit. For example, a remote user, such as a vendor representative, may take control of the sensor (e.g., camera). The remote user may be able to pan, tilt, or zoom the sensor. In one example, a vendor representative may wish to take a closer look at a vendor's shelf space. The remote user may be able to live-stream the information captured by the sensor. For example, the remote user may view a live image that is captured by one or more camera. The remote user may control the sensor as desired to collect more information about the items. If the sensor is operating on a schedule, the schedule may be interrupted to accommodate requests by the remote user to access the sensor. In some instances, if multiple remote users wish to take control of the sensor, the first remote user that put in the request may be able to access the sensor first. The second remote user may be able to take control when the first remote user is finished using the sensor. In other instances, there may be priority levels or hierarchies granted to remote users. Remote users that are higher up in the hierarchy may be able to access the remote sensor first and/or have precedence.

In some instances, depending on the identity of the remote user, the remote user may only have limited control or access to the information. For example, if the remote user is a vendor representative, the vendor representative may only be able to access information relating to the shelf space that is stocked by the corresponding vendor. For example, if Vendor X stocks shelves 1, 3, and 4, Vendor X may only be able to control the sensor to be directed to shelves 1, 3, and 4, without being able to access information on shelves 2 and 5. If Vendor Y stocks shelves 2 and 5, Vendor Y may only be able to control the sensor to be directed to shelves 2 and 5 without being able to access information on shelves 1, 3, and 4. Alternatively, any remote user may be able to direct the sensor in any manner. A remote user identity may be discerned and/or authenticated prior to granting access to sensor data and/or control. The remote user identity may have an associated access level or parameters that may be followed when granting the remote user access.

The remote user may be able to view the sensor data on the user interface. Controls for controlling the sensor may optionally be provided on the same user interface. For example, the user may swipe a touchscreen to control a direction that a sensor is facing. The user may be able to pinch in or out to zoom in or out. Other controls may be provided on the user interface or through additional user interactive devices (e.g., joysticks, trackballs, touchpads, etc.). The systems and methods provided herein may provide a portal for a vendor or other remote user to access the real-time information.

Allow remote users access and/or control may advantageously allow remote users to remotely monitor the inventory and only make a physical trip when necessary. This may save time for vendor representatives who would otherwise physically visit the facility on a regular basis.

The sensor may have the necessary characteristics to detect the indicators of various items. For instance, a camera may have a sufficient resolution to be able to discern visual indicators from the camera's location. For example, the camera may be capable of at least capturing images at least a 6 mega pixel, 8 mega pixel, 10 mega pixel, 12 mega pixel, 20 mega pixel, 4K, or 8K resolution. The camera may utilize light balancing to obtain a desired level of contrast. The indicators may be arranged on the item so that they can be detected by the sensor. For example, if the sensor is on a wall opposite a storage unit, visual indicators may be arranged to face the wall that supports the camera so that the visual indicators are viewable by the camera. In another example, if a camera is supported on a top surface of a shelving unit, the visual indicators may be arranged on top of the items so that they can be viewable by the camera.

The sensor may be able to discern an indicator sufficiently to collect the relevant information. As previously described, the information may be encoded into the indicator itself, or may be associated with the indicator. In some embodiments, the sensor may be able to visually discern indicators. Optionally, barcode or QR code reading or scanning may occur with aid of the sensor. In some instances, image recognition techniques may be utilized. For example, the indicator may include a label with text or images. Optical character recognition or other techniques may be used to collect data from the indicator. In some instances, object recognition platforms may be utilized. For instance, an image of the indicator or the item itself may be compared with one or more known objects. In some instances, feature point extraction may be utilized to analyze images captured by a camera. Any machine vision techniques known or later developed in the art may be utilized to aid in indicator recognition and discernment.

The sensor may have the necessary characteristics to detect a level of supply for various items. For instance, the sensors may be able to detect how many units, batches, or groups of items remain in the storage unit. The sensor may be able to get a sense of supply on the order of at least a single item, 5 items, 10 items, 20 items, 30 items, 50 items, 100 items, 200 items, or 500 items.

In some embodiments, one or more sensors may be able to detect that the supply for a particular item has fallen beneath a certain threshold. Similarly, one or more sensors may aid in collecting data that may be used to determine whether particular items are about to expire within a certain predetermined period of time. An alert or message may be automatically sent to particular parties. For instances, alerts or messages may be sent to a corresponding vendor or vendor representative. Similarly, alerts or messages may be sent to particular health care provider administrators or employees.

As previously described, one or more sensors may be located or supported outside the storage unit and be able to capture information about items within the storage unit. In some instances, one or more sensors may be supported by a wall, top surface, bottom surface, shelf, or other portion of a storage unit. A sensor may be integrally built into a storage unit or may be removable from a storage unit. A sensor may or may not need to be within a predetermined proximity of an item to detect the item and/or discern the indicator for an item.

Per FIG. 2, the first sensor 210 may be located proximate to the storage unit 240 in a position that enables capture of the indicators of the first item 201, the second item 202, or both. The second sensor 220 may be located on the storage unit 240 in a position that enables capture of the indicators of the first item 201, the second item 202, or both. The display 230 may be located on or proximate to the storage unit 240.

FIG. 3 shows an exemplary embodiment, when the sensor is utilized at a location where a medical procedure may take place. In some instances, the sensors may be used for additional functions beyond inventory management. For instance, the same sensor may be used track a surgical procedure or surgical site, and also to keep track of inventory. Alternatively, different sensors may be used for inventory management and for the medical procedure. The sensors may optionally be supported by a medical console, the storage unit, or a portion of the room, such as a wall, ceiling, or furniture.

Per FIG. 3, the first sensor 210 may be located proximate to the display 230 in a position that enables capture of the indicators of the first item 201, the second item 202, or both. The second sensor 220 may be located on a tool table 340 in a position that enables capture of the indicators of the first item 201, the second item 202, or both. The display 230 may be located on or proximate to the storage unit 240.

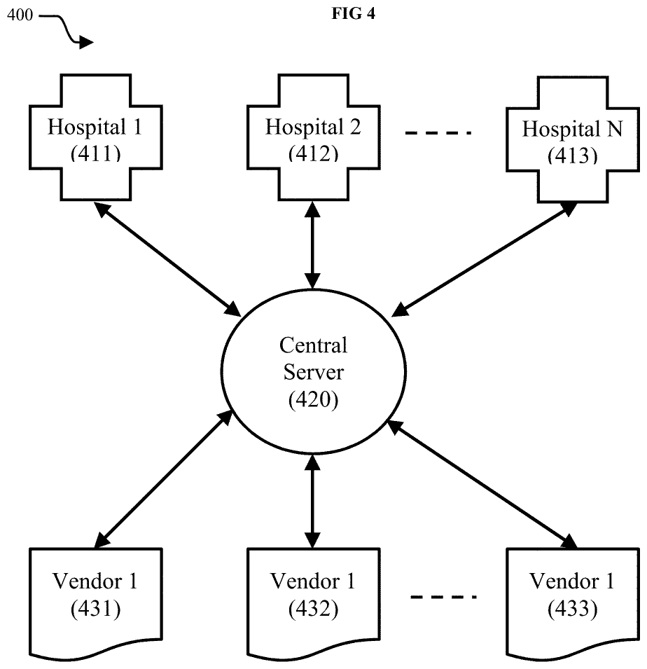

FIG. 4 shows a non-limiting schematic diagram of a real-time inventory capture system. The systems, methods, and applications 400 herein may employ a central server 420 configured for real-time cumulative inventory tracking throughout various medical facilities or hospitals 411 412 413. The cumulative inventory tracking may be communicated simultaneously to two or more vendors 431 432 433. Such a central server 420 may additionally perform inventory optimization through algorithms and machine learning. Any machine learning technique, such as those described elsewhere herein, may be employed.

Any description herein of a central server may apply to one or more servers, cloud computing infrastructure, or any other type of infrastructure. The central server may be owned or operated by an entity that manages the real-time inventory information. This collection of information from multiple facilities may provide a wider view of inventory optimization that may take place. While vendors are provided by way of example, other parties may have access to real-time information, such as a health care facility, administrator, group, practitioner, vendor or vendor representative, insurance, manufacturers, or other data collection systems. In some instances, various parties may have limited access to the real-time information. For example, the vendors may be provided with access to the information pertaining to the vendors. In some instances, industry-wide information may be analyzed and/or made available to various parties.

Providing access to the real-time product usage and inventory information may advantageously improve business process interfacing with health care facilities. This may improve or optimize inventory predictions for health care facilities, regions, countries, and allow for vendors and manufacturers to adjust supply. This may also reduce the amount of shelf space needed, since better predictions may allow shelf space to be utilized more effectively. The systems and methods provided herein may also be able to avoid expired inventory, which may reduce overall waste.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

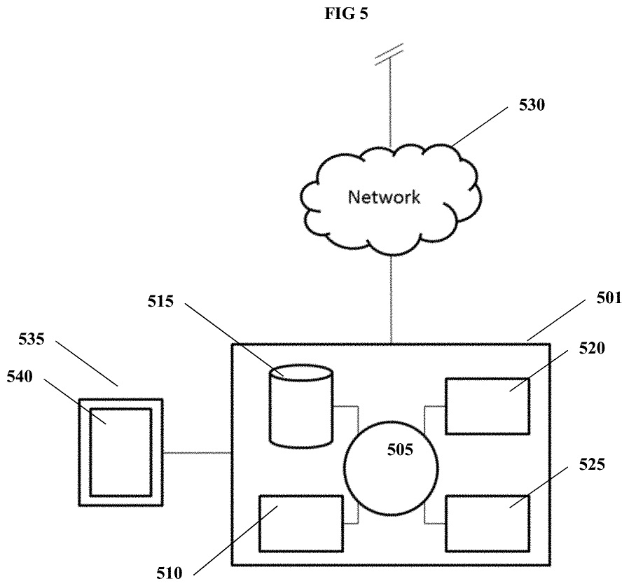

Referring to FIG. 5, in a particular embodiment, a digital processing device 501 is programmed or otherwise configured to create an enterprise resource planning application. The device 501 is programmed or otherwise configured to create an enterprise resource planning application. In this embodiment, the digital processing device 501 includes a central processing unit (CPU, also “processor” and “computer processor” herein) 505, which is optionally a single core, a multi core processor, or a plurality of processors for parallel processing. The digital processing device 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 comprises a data storage unit (or data repository) for storing data. The digital processing device 501 is optionally operatively coupled to a computer network (“network”) 530 with the aid of the communication interface 520. The network 530, in various cases, is the internet, an internet, and/or extranet, or an intranet and/or extranet that is in communication with the internet. The network 530, in some cases, is a telecommunication and/or data network. The network 530 optionally includes one or more computer servers, which enable distributed computing, such as cloud computing. The network 530, in some cases, with the aid of the device 501, implements a peer-to-peer network, which enables devices coupled to the device 501 to behave as a client or a server.

Continuing to refer to FIG. 5, the CPU 505 is configured to execute a sequence of machine-readable instructions, embodied in a program, application, and/or software. The instructions are optionally stored in a memory location, such as the memory 510. The instructions are directed to the CPU 105, which subsequently program or otherwise configure the CPU 505 to implement methods of the present disclosure. Examples of operations performed by the CPU 505 include fetch, decode, execute, and write back. The CPU 505 is, in some cases, part of a circuit, such as an integrated circuit. One or more other components of the device 501 are optionally included in the circuit. In some cases, the circuit is an application specific integrated circuit (ASIC) or a field programmable gate array (FPGA).

Continuing to refer to FIG. 5, the storage unit 515 optionally stores files, such as drivers, libraries and saved programs. The storage unit 515 optionally stores user data, e.g., user preferences and user programs. The digital processing device 501, in some cases, includes one or more additional data storage units that are external, such as located on a remote server that is in communication through an intranet or the internet.

Continuing to refer to FIG. 5, the digital processing device 501 optionally communicates with one or more remote computer systems through the network 530. For instance, the device 501 optionally communicates with a remote computer system of a user. Examples of remote computer systems include personal computers (e.g., portable PC), slate or tablet PCs (e.g., Apple® iPad, Samsung® Galaxy Tab, etc.), smartphones (e.g., Apple® iPhone, Android-enabled device, Blackberry®, etc.), or personal digital assistants.

Methods as described herein are optionally implemented by way of machine (e.g., computer processor) executable code stored on an electronic storage location of the digital processing device 101, such as, for example, on the memory 510 or electronic storage unit 515. The machine executable or machine readable code is optionally provided in the form of software. During use, the code is executed by the processor 505. In some cases, the code is retrieved from the storage unit 515 and stored on the memory 510 for ready access by the processor 505. In some situations, the electronic storage unit 515 is precluded, and machine-executable instructions are stored on the memory 510.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

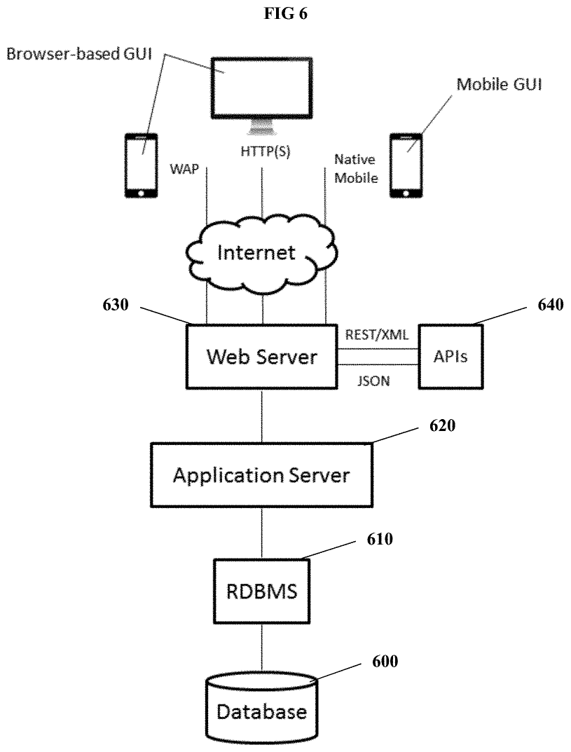

Referring to FIG. 6, in a particular embodiment, an application provision system comprises one or more databases 600 accessed by a relational database management system (RDBMS) 610. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 620 (such as Java servers, .NET servers, PHP servers, and the like) and one or more web servers 630 (such as Apache, IIS, GWS and the like). The web server(s) optionally expose one or more web services via app application programming interfaces (APIs) 640. Via a network, such as the internet, the system provides browser-based and/or mobile native user interfaces.

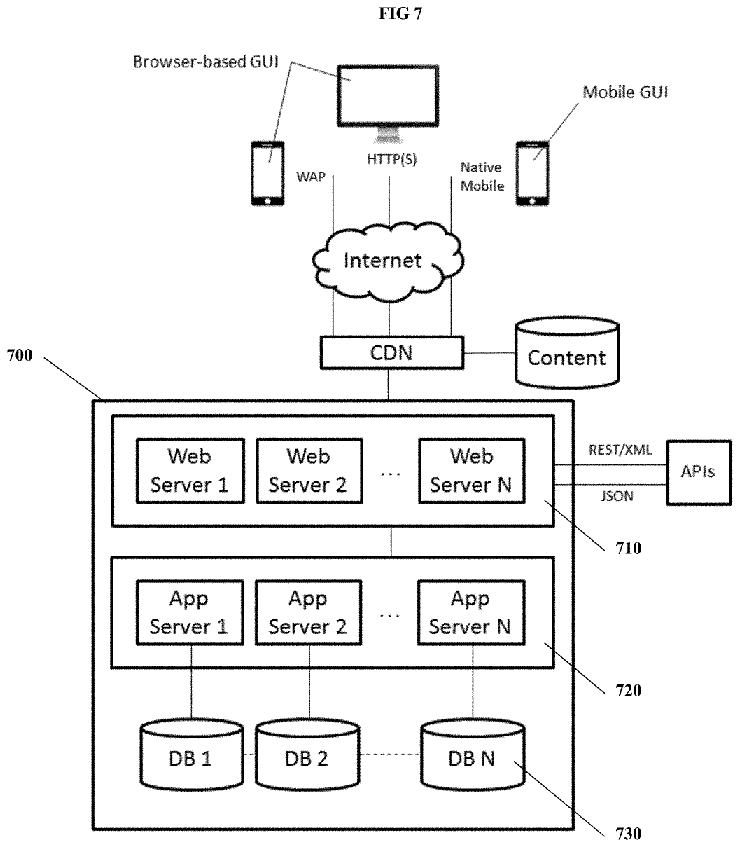

Referring to FIG. 7, in a particular embodiment, an application provision system alternatively has a distributed, cloud-based architecture 700 and comprises elastically load balanced, auto-scaling web server resources 710 and application server resources 720 as well synchronously replicated databases 730.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for creating an enterprise resource planning application. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### EXAMPLES

The following illustrative examples are representative of embodiments of the software applications, systems, and methods described herein and are not meant to be limiting in any way.

### Example 1—Stent Device Selection

Stents A and B are both carried by hospital X and are equivalent for use in procedure Y. The hospital has currently used quantity 50 of Stent A, which costs $1.00 per stent, and quantity 99 of Stent B, which costs $5.00 per stent for purchased quantities of 0-99 and $0.75 per stent for purchased quantities of 100 and above. The medical professional is familiar with Stent A and selects it for procedure Y. The inventory module receives the practitioner item selection of Stent A by a captured image of the stock room. The equivalence notification module then displays an equivalence notification to select Stent B based on its lower price, and the ability to reach the pricing threshold. The medical practitioner then replaces Stent A with Stent B and the inventory module receives the updated practitioner item selection of Stent B by a subsequent captured image of the stock room. The reporting module receives the practitioner's updated response updates Stent B's item stock, Stent B's utilized quantity. The reporting module also sends a notification based on or comprising the updated response to a vendor associated with Stent A, an administrator of hospital Y, or both.

### Example 2—Stent Device Selection

Stents A and B are both carried by hospital X and are equivalent for use in procedure Y. The medical professional is familiar with Stent A and selects it for procedure Y. The inventory module receives the practitioner item selection of Stent A by a captured barcode of Stent A. The equivalence notification module then determines if an equivalence notification should be made for Stent B based on the price structure or contractual terms and the utilized quantities of Stent A and B. As Stent A and B were determined to be medically equivalent by the administration of hospital X but the quantity of Stent A that has been purchased and used by hospital X has not met a price structure or contractual term milestone. As such, an equivalence notification is not displayed.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A medical resource planning system comprising:
a memory storage unit configured to store data pertaining to a first item and a second item, wherein (1) the first item associated with a first price structure or contractual term, a first item stock, a first item utilized quantity, and an equivalence association with the second item, and (2) the second item associated with a second price structure or contractual term, a second item stock, a second item utilized quantity, and an equivalence association with the first item;
an inventory module configured to, with aid of one or more processors, receive and recognize a practitioner item selection as the first item with aid of a sensor configured to detect the practitioner item selection; and
a display device configured to display an equivalence notification to a practitioner based on the equivalence association of the first item with the second item, and when a predetermined relation is reached between the one or more of the first price structure or contractual term and the second price structure or contractual term, the first item stock and the second item stock, or the first item utilized quantity and the second item utilized quantity.

2. The system of claim 1, further comprising an input device configured to receive a practitioner response to the equivalence notification and updating the practitioner item selection data based on the practitioner response.

3. The system of claim 2, further comprises a reporting module receiving the practitioner response and updating at least one of the first item stock, the second item stock, the first item utilized quantity, and the second item utilized quantity based on the practitioner response.

4. The system of claim 1, wherein at least one of the first item and the second item comprise a medical tool, an instrument, an implant, a medicine, an ointment, a disposable, or a practitioner device.

5. The system of claim 1, wherein the sensor comprises a camera, a video camera, an RFID reader, a radar, a sonar, a LIDAR, a thermal imaging camera, a scale, a button, or any combination thereof.

6. The system of claim 1, wherein the sensor comprises a camera and the practitioner item selection is recognized as the first item based on image recognition of the practitioner item selection.

7. The system of claim 1, wherein the sensor comprises a camera and the practitioner item selection is recognized based on a visual graphical code on the practitioner item selection or packaging associated with the practitioner item selection.

8. The system of claim 1, wherein the inventory module further receives a practitioner item confirmation, a practitioner item note, or both, wherein the practitioner item confirmation and the practitioner item note are associated with the practitioner item selection.

9. The system of claim 1, wherein at least one of the first price structure or contractual term and the second price structure or contractual term comprise at least a primary cost per item for a primary quantity of items and a secondary cost per item for a secondary quantity of items.

10. The system of claim 1, wherein at least one of the first price structure or contractual term and the second price structure or contractual term comprise a set price per part per quantity.

11. The system of claim 1, wherein the equivalence notification comprises the first price structure or contractual term, the second price structure or contractual term, the first item stock, the second item stock, the first item utilized quantity, the second item utilized quantity, or any combination thereof.

12. The system of claim 1, wherein the equivalence notification comprises a difference between the first price structure or contractual term and the second price structure or contractual term, the first item stock and the second item stock, the first item utilized quantity and the second item utilized quantity, or any combination thereof.

13. The system of claim 1, wherein the equivalence notification comprises a quantity of the first item or the second item necessary to reach the secondary cost per item.

14. The system of claim 1, wherein the equivalence notification comprises an interchangeability rating based on a historical usage data of the first item and the second item.

15. The system of claim 1, wherein at least one of the first price structure or contractual term and the second price structure or contractual term comprise a lack of a price structure or contractual term.

16. The system of claim 1, wherein the equivalence notification is further associated with contract met status based on the first price structure or contractual term and the first item utilized quantity, or the second price structure or contractual term and the second item utilized quantity.

17. A computer-implemented medical resource planning method comprising:
storing, at a memory storage unit, data pertaining to a first item and a second item, wherein (1) the first item associated with a first price structure or contractual term, a first item stock, a first item utilized quantity, and an equivalence association with the second item, and (2) the second item associated with a second price structure or contractual term, a second item stock, a second item utilized quantity, and an equivalence association with the first item;
receiving and recognizing, at an inventory module with aid of one or more processors, a practitioner item selection as the first item with aid of a sensor configured to detect the practitioner item selection; and
displaying, on a display device, an equivalence notification to a practitioner based on the equivalence association of the first item with the second item, and when a predetermined relation is reached between the one or more of the first price structure or contractual term and the second price structure or contractual term, the first item stock and the second item stock, or the first item utilized quantity and the second item utilized quantity.

18. The method of claim 17, further comprising receiving, via an input device, a practitioner response to the equivalence notification and updating the practitioner item selection data based on the practitioner response.

19. The method of claim 17, wherein at least one of the first item and the second item comprise a medical tool, an instrument, an implant, a medicine, an ointment, a disposable, or a practitioner device.

20. The system of claim 17, wherein the sensor comprises a camera and the practitioner item selection is recognized as the first item based on (a) image recognition of the practitioner item selection, or (b) a visual graphical code on the practitioner item selection or packaging associated with the practitioner item selection.
